(19) United States
(12) Patent Application Publication
(10) Pub. No.: US 2020/0245890 A1
(43) Pub. Date: Aug. 6, 2020

(54) BIOFEEDBACK SYSTEM AND WEARABLE DEVICE

(71) Applicant: Thought Beanie Limited, Bristol (GB)

(72) Inventors: Simon HARRISON, Bristol (GB); Alyn MORGAN, Bristol (GB)

(73) Assignee: Thought Beanie Limited, Bristol (GB)

(21) Appl. No.: 16/633,818

(22) PCT Filed: Jul. 23, 2018
(86) PCT No.: PCT/EP2018/069925

§ 371 Date: Jan. 24, 2020

(30) Foreign Application Priority Data
Jul. 24, 2017 (GB) 1711887.8

(51) Int. Cl. A61B 5/0482; A61B 5/00; A61B 5/0478; A61B 5/16; A61B 5/01; A61B 5/021; A61B 5/053; A61B 5/08; A61B 5/145

(52) U.S. Cl. CPC A61B 5/0482; A61B 5/0006; A61B 5/0478; A61B 5/165; A61B 5/6803; A61B 2562/164; A61B 5/021; A61B 5/0533; A61B 5/08; A61B 5/14507; A61B 2503/10; A61B 5/01

(57) ABSTRACT

A biofeedback system capable of obtaining a real-time EEG response “in the field”, i.e. while a user is performing an activity in a real-world (non-clinical) setting, and capable of transforming the EEG response into a meaningful indicator of current mental state, and presenting that indicator to the user, e.g. in a form able to improve their performance of the activity. The system comprises a wearable sensor incorporated into headgear worn by the user during participation in an activity. A central processing unit is arranged to receive an EEG signal transmitted from the wearable sensor, and filter and analyse the EEG signal to generate output data that is indicative of mental state information for the user.

### CROSS REFERENCE TO RELATED APPLICATIONS

This is a U.S. National Phase Application under 35 U.S.C. § 371 of International Patent Application No. PCT/EP2018/069925, filed Jul. 23, 2018, which claims priority of United Kingdom Patent Application No. 1711887.8, filed Jul. 24, 2017. The entire contents of which are hereby incorporated by reference.

### FIELD OF THE INVENTION

The invention relates to a system for detecting an electroencephalographic (EEG) response from a user in real time while the user participating in an activity, e.g. a sporting activity, fitness assessment, or the like. In particular, the invention relates to a system in which a dedicated EEG signal can be used to provide neurofeedback, augmented by the potential for wider biofeedback, for the user.

### BACKGROUND TO THE INVENTION

Wearable technology for monitoring physiological properties of a user during an activity is a recent and popular phenomenon. Wearable sensors may be self-contained, or may interface with other accessories, such as smartphones, smartwatches, tablet computers or the like. Collected information may be used to monitor performance and influence training, etc.

More recently, there is an interest in monitoring mental activity (e.g. emotional state) as a means of understanding or improving user performance. It is known that a user's electroencephalographic (EEG) response can reliably be used to assess and improve sporting performance. However, to date such assessments are typically performed in a laboratory or clinical setting, using equipment that is either too unwieldy or too expensive to be released as a consumer offering.

### SUMMARY OF THE INVENTION

At its most general, the present invention provides a biofeedback system capable of obtaining a real-time EEG response “in the field”, i.e. while a user is performing an activity in a real-world (non-clinical) setting, and capable of transforming the EEG response into a meaningful indicator of current mental state, and presenting that indicator to the user, e.g. in a form able to improve their performance of the activity. An independent aspect of the system presented herein is a wearable sensor that can be incorporated (e.g. integrally formed with or mounted within) existing conventional headwear, e.g. sports headwear, such as a cap, a helmet, etc. The wearable sensor may be configured with a multi-channel sensing unit arranged to wirelessly communicate with a base station processing unit, which may be a smartphone, tablet computer or other portable computing device.

According to a first aspect of the invention, there is provided a wearable sensor for measuring an electroencephalograph (EEG) response from a user's scalp, the wearable sensor comprising: headgear to be worn by a user while participating in an activity; a sensor array mounted in the headgear, the sensor array comprising a plurality of sensor elements for making electrically conductive contact with a user's scalp; a detector mounted in the headgear, the detector being arranged to detect voltage fluctuations at each of the plurality of sensor elements and generate an EEG signal therefrom; and a transmitter for wirelessly transmitting the EEG signal to a remote device for analysis, wherein the plurality of sensor elements are disposed within the headgear to lie across the skull of the user when the headgear is worn. This aspect of the invention may thus provide device capable of use in real-world scenarios, e.g. when a user is engaged in a sport or other game, to provide an EEG signal that is indicative of fear/anxiety and confidence/excitement, which are understood to have a polarising effect on athletic performance. As explained below, the EEG signal can be analysed remotely to provide feedback about the user's mental state during performance of the activity, which in turn can be used to assess and/or improve that performance.

As mentioned above, the headgear may be any type of headgear that is worn by the user while engaging the activity. The headgear may specific to the activity. For example, the headgear may be a baseball cap, a crash helmet, a sport helmet, and a swimming cap. The headgear may be compulsory for participating in the activity (e.g. crash helmet). The invention may differ from conventional EEG sensors in being incorporated directly into the specific type of headgear normally worn when participating in an activity.

The headgear may be any suitable article for wearing on the user's head. It can be a cap, helmet, protective mask or the like.

The sensor array may desirably be located over the user's frontal lobe, the headgear may have a frontal lobe cover portion to which the sensor array is attached. The plurality of sensor elements may protrude from an inner surface of the headgear to contact the user's scalp. However, the detector and transmitter may be incorporated or integrally formed within the material of the headgear.

The detector and transmitter may be provided together in a single control unit. The control unit may also include a battery and processor for controlling the device. The control unit may be mounted on a flexible substrate (e.g. flexible circuit board) that conforms to the shape of the headgear. The detector may be arranged to convert the voltage fluctuations into a digital signal suitable for transmission. The detector may be arranged to receive voltage signals from the plurality of sensor elements over a plurality of channels, e.g. by multiplexing between the sensor elements. For example, the plurality of sensor elements may be located at any one or more of the FP_{z}, FC₅, FC₆, C_{z}, AF₇, AF₈ and FC_{z} positions across the frontal lobe. Detecting signals from a plurality of sensor locations can improve the accuracy of the resulting EEG signal.

The sensor may comprise a conductive interconnection structure formed within the headgear to provide an electrical connection between the sensor array and the detector. The conductive interconnection structure may comprise a conductive fabric sandwiched between a pair of insulation layers. The insulation layers can reduce or minimise interference on the signal received at the detector. The conductive interconnection structure may be encased within the material of the headgear.

Each sensor element may comprise a star-shaped body having a plurality of resiliently deformable legs that extend radially from a central portion. When the headgear is mounted on the user's head, the legs push outwards to move away hair from the sensor element location and facilitate a good electrical contact with the scalp. The central portion of the star-shaped body may be electrically conductive and arranged to come into physical contact with the user's scalp when the headgear is worn. The star-shaped body may be made from a lightweight material such as graphite or the like for improved comfort. A micro-electrode with high conductivity (e.g. made from gold of the like) may provide an electrical connection between the star-shaped body and the conductive interconnection structure within the headgear.

The sensor may include an amplification module arranged to amplify the signals received from the sensor array before they are transmitted.

The transmitter may be arranged to wirelessly transmit the EEG signal over any suitable network. In one example, the transmitter may operate over a WiFi network to send the EEG signal to a network-enabled computing device (e.g. a smartphone, smartwatch, tablet computer or the like). In another example, the transmitter may be paired with a remote device over a short range wireless network (e.g. Bluetooth®) to transmit the EEG signal.

In another aspect, the invention provides a biofeedback system comprising: a wearable sensor comprising: a sensor array for detecting an electroencephalographic (EEG) signal from a user wearing the wearable sensor; a communication unit for wirelessly transmitting the EEG signal; and a central processing unit arranged to receive the EEG signal transmitted from the head-mountable wearable sensor, the central processing unit comprising an analyser module arranged to generate, based on the EEG signal, output data that is indicative of mental state information for the user, wherein the wearable sensor is incorporated into headgear worn by the user during participation in an activity, whereby the output data provides real-time mental state information for the user whilst performing the activity. In this aspect, the invention provides a computing device that may be capable of generating, in real-time, output data that is indicative of a user's mental state when performing an activity.

The wearable sensor may have any of the properties or features discussed above with respect to the first aspect of the invention.

The output data may be based on an analysis of the EEG signal. In one example, the analysis may comprise applying the EEG signal to a model that extracts features therefrom and maps them to output data, e.g. in the form of a vector, that is indicative of a user's mental state. The model may be based on an suitable algorithm that has been trained by machine learning or similar techniques. The format of the output data may take any suitable form. However, in one example, the output data may relate the EEG signal to an individual zone of optimal functioning (IZOF) model for the user.

The headgear may comprise any of a baseball cap, a crash helmet, a sport helmet, and a swimming cap.

The output data may be presented in a graphical manner on a display associated with the central processing unit. In one example, the central processing unit is part of a portable computing device, such as a smartphone, tablet computer or the like. The output data may be presented on this device.

To improve the accuracy of the output data, the central processing unit may comprise a filter module for removing unwanted and/or irrelevant frequencies from the EEG signal before it is used to generate the output data. The unwanted and/or irrelevant frequencies may relate to interference. The filter module may operate to extract desired EEG frequency bands from the EEG signal. For example, the Alpha and Theta bands may be of particular interest.

Advantageously, the central processing unit may be arranged to receive biometric data for the user concurrently with the EEG signal. The analyser module may be arranged to use the biometric data to inform or assist in the generation of the output data based on the EEG signal. For example, the output data may be based on a combination of the EEG signal and biometric data. Or the biometric data may be used to cross-check the output data. In one example, the biometric data may be used to fine-tune the model used to generate the output data.

The biometric data may be obtained from other wearable devices associated with (worn by) the user. For example, the biometric data may be sent from sensors integrated into clothing, body straps, legbands, wristbands or the like. The biometric data may include any one or more of breathing patterns, heart rate, blood pressure, skin temperature, galvanic skin response, and salivary cortisol (e.g. from a post-activity spit test).

The central processing unit may also receive additional user-related data, e.g. concerning motion, behaviour and position during the activity. This information can be used to further inform the output data or can be synchronised with the EEG signal or output data to provide feedback about the circumstances in which certain mental states occur. In some examples, the central processing unit may receive audio and/or video data of the user performing the activity. This information may be used in conjunction with the output data to provide feedback to the user after the activity has concluded.

The central processing unit may comprise a correlator module arranged to correlate the other data mentioned above with the EEG signal. As mentioned above, the correlation may be for the purpose of refining or checking the output data resulting from the EEG signal.

The wearable sensor and biofeedback system disclosed herein provide a readily accessible tool for facilitating mental training of a user engaging in a certain activity. It can be used as a data source for the subsequent provision of neurofeedback, for example.

### BRIEF DESCRIPTION OF THE DRAWINGS

Embodiments of the invention are described in detail below with reference to the accompanying drawings, in which:

FIG. 1 is a schematic view of a biofeedback system that is an embodiment of the invention;

FIG. 2 is a schematic view of a portable processing unit for mounting in a wearable article;

FIG. 3A is a perspective view of an electroencephalographic (EEG) sensor unit that is suitable for use with the invention;

FIG. 3B is a side view of the EEG sensor unit in contact with a user's scalp;

FIG. 4A is a schematic plan view of a user's head showing an EEG sensor array configuration suitable for use with the invention;

FIG. 4B is a chart showing a result of mapping an EEG signal for a user to a set of performance emotions;

FIG. 5 is a schematic view of a system according to the invention being using during user activity;

FIG. 6 is a schematic view of a wearable unit that can be used in a first embodiment of the invention;

FIG. 7 is a schematic view of a wearable unit that can be used in a second embodiment of the invention; and

FIG. 8 is a schematic view of a wearable unit that can be used in a third embodiment of the invention.

### DETAILED DESCRIPTION

The present invention relates in general to a biofeedback system in which an electroencephalographic (EEG) signal (often referred to as brainwaves) is detected for a user whilst the user is performing an activity (such as a sport) for the purpose of aiding understanding of and facilitating improvement of the user's mental state when performing that activity. The EEG signal alone or in combination with other biometric data may be mapped on to a representation of associated mental states, e.g. concerning concentration, stress, etc. This information in turn can be indicative of or used to boost or otherwise improve the user's individual zone of optimal function (IZOF), e.g. by making user of known techniques in the field of neurofeedback.

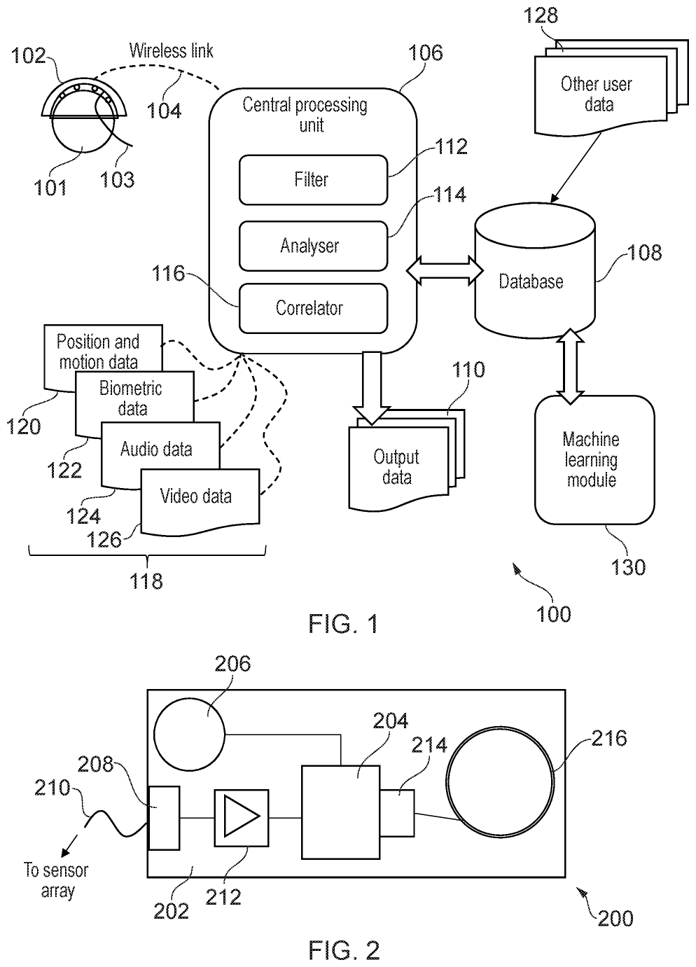

FIG. 1 is a schematic diagram of a biofeedback system 100 that is an embodiment of the invention. In simple terms, the system 100 comprises three components: (i) a wearable sensor, which may be incorporated into a piece of sports equipment (e.g. helmet) or sportswear (e.g. baseball cap); (ii) a processing unit, which may be smartphone, smartwatch, tablet or other computing device communicably connected to the wearable sensor; and (iii) a database or other storage or memory facility in communication with the processing unit to provide information that assist analysis of data from the wearable sensor. The three components may be separate from one another or may be located together, in any combination. Similarly, the functions of the processing unit described below may be performed by a plurality of processors in different locations. The processing and/or analysis may thus occur locally, e.g. at a processing unit in the same location as the user, or remotely, e.g. at a processing unit in the cloud or the like.

In FIG. 1, the system 100 comprises a head-mountable wearable device 102 on a user's head 101. As discussed above, the wearable device 102 may be any suitable piece of headwear used when a user performed an activity. A wearable sensor module 103 is mounted or otherwise incorporated or integrated within the headwear. Advantageously, the wearable sensor module of the present invention may be mounted within a standard piece of sports equipment or sportswear, which makes the invention readily available for use in real scenarios, rather than only in laboratory conditions. Some examples of this are discussed below.

The wearable sensor module 103 comprises a sensor array comprising a plurality of sensor elements for obtaining an electroencephalographic (EEG) signal from a user while wearing the headwear. Each sensor element may be arranged to contact the user's scalp to obtain a suitable measurement. The plurality of sensor elements may be located within the headwear at suitable positions for obtaining an EEG signal from suitable nodes across the user's skull. The location of the sensor elements may be selected to facilitate detection of a set of predetermined emotions that are relevant to the activity. For example, the set of predetermined emotion may relate to any one or more emotions that influence athletic performance, such as fear, anger, confidence, concentration, focus, etc. In one example, the sensors are located across the frontal lobe of the user.

The wearable sensor module 103 includes a local processing unit (an example of which is shown in FIG. 2), for controlling the sensor array and generating an EEG signal based on readings from the sensor array. The wearable sensor module 103 may be equipped with a wireless transmitter for transmitting the EEG signal to a remote central processing unit 106 for further processing. The wireless transmitter may send the signal over any suitable network using any suitable protocol, e.g. WiFi, Bluetooth®, etc. The wireless transmitter may include 4G or 5G connectivity for immediate transmission and real-time response.

In other examples, the wearable sensor module may include a storage unit, e.g. a computer writable memory such as flash memory or the like, where information can be stored in the headwear and then downloaded and analysed later (e.g. via a wired link). This may be useful where the activity being performed limits or prevents wireless connectivity, e.g. sailing, swimming, cycling etc.

The central processing unit 106 is a computing device used to analyse and report on the EEG signal. Any computing device capable of receiving the EEG signal from the wearable sensor module may be used. For example, the central processing unit 106 may be a smartphone, tablet computer, laptop computer, desktop computer, server computer or the like. The central processing unit 106 comprises a memory and a processor for executing software instructions to perform various functions using the EEG signal. In the example illustrated in FIG. 1, the central processing unit 106 is shown to have three modules that perform different functions.

The central processing unit 106 comprises a filter module 112 arranged to clean up the received EEG signal, e.g. by filtering out environmental artefacts and/or other unwanted frequencies. For example, the filter module may be arranged to extract data correspond to target EEG bands from the obtained EEG signal. The target EEG bands may, amongst others, comprise the Alpha and Theta bands (8-15 Hz and 4-7 Hz respectively).

The central processing unit 106 comprises an analyser module 114 that is arranged to process the EEG signal (e.g. after filtering by the filter module 112) to yield information indicative of the user's mental state. For example, the information may be indicative or an emotional state of the user, and/or may provide an objective measurement of a current mental process, e.g. concentration, stress, relaxation, etc. The analyser module 114 may be configured to process the (filtered) EEG signal in a manner such that the mental state information is effectively generated in real time. To generate the mental state information discussed above, the analyser module 114 may be configured to map the EEG signal onto a mental state vector, whose components are each or are each indicative of an intensity value or probability for a respective emotional state or mental process. The mapping process may be based on a suitable software model drawing on machine learning and artificial intelligence. The analyser model may be adaptive to an individual's responses. In other words it may learn to recognise how an individual's detected EEG signals map on to emotional state information. This can be done through the use of targeting sampling and predictive AI techniques. As a result, the analyser module may improve in accuracy and responsiveness with use.

In one specific example, the analyser module 114 may measure asymmetry in the Alpha (confidence) and Beta (composure) EEG bands across the left hemispheric bank to determine positive emotion and make corresponding measurements over the right hemisphere to measure the opposite. An output from this analysis can be indicative of negative anxiety/stress activation in the right prefrontal cortex, amygdala, and insula. Furthermore, the analyser module may be arranged relate the EEG signal (or the output that results from the analysis thereof) to an individual zone of optimal functioning (IZOF) model for the user. This information may be included in an output from the central processing unit 106, e.g. in the form or a graphical display or data transmission, that can be used to assist in optimising the user's performance in the activity being undertaken.

The mental status information from the analyser module 114 may be transmitted to a repository (e.g. a database 108) where is can be aggregated with data 128 from the other users to form a dataset that can be in turn be used to inform and improve the analysis algorithm, e.g. via a machine learning module 130 that may train a model based on aggregated data in the database 108.

The central processing unit 106 may comprise a correlator module 116 that is arranged to correlate or synchronise the EEG signal with other user-related data 118 received at the central processing unit 106. The correlator module 116 may operate to combine the EEG signal with other data before it is processed by the analyser module 114. The other data may include biometric data 122 recorded for the user, e.g. from other wearable devices that can interface with the central processing unit 106. The biometric data 122 may be indicative of physiological information, psychological state or behavioural characteristics of the user, e.g. any one or more of breathing patterns, heart rate (e.g. ECG data), blood pressure, skin temperature, galvanic skin response (e.g. sweat alkalinity/conductivity), and salivary cortisol (e.g. obtained from a spit test). In one example, the correlator module may be arranged to correlate an imbalance between sympathetic and parasympathetic arms of the autonomic nervous system as indicated by the other user-related data.

In some examples, the analysis performed by the analyser module 114 may utilise a range of different physiological and mental responses. This may improvement the accuracy or reliability of the output data. For example, the biometric data may be used to sense check the mental state information obtained from the EEG signal. Moreover, the biometric data may be stored in conjunction with the mental state information in the database 108 to provide a profile for the user, i.e. a personal history or record of measured mental and physiological response during performance of an activity. The analyser module 114 may be arranged to refer to the profile as a means of refining a measurement. Similarly, the analyser module 114 may be arranged to access an aggregated profile from the database as a means of providing an initial baseline with which to verify or calibrate measurements for a new user.

The other user-related data 118 may include information relating to the activity being performed by the user to assist in matching the user's mental state to specific situations in the activity. For example, the other user-related data 118 may include position and/or motion data 120. The position data may be acquired from a global position system (GPS) sensor or other suitable sensors, and may be used to provide information about the location of the user during the activity, e.g. the location on a playing surface, such as a pitch, court, track, etc. The motion data may be from a motion tracker or sensor, e.g. a wearable sensor, associated with the user. The motion data may be acquired from accelerometer, gyroscopes or the like, and may be indicative or the type and/or magnitude of movement or gesture being performed by the user during the activity. The correlator module 116 of the central processing unit 106 may be able to match or otherwise link the EEG signal with the position data and/or motion data to perform information on physical characteristics of the user whilst exhibiting the observed mental state. This information may be used to provide feedback to the user to improve performance.

The other user-related data 118 may include audio data 124 and/or video data 126 recorded for the user. This information may effectively be an enhanced version of the position and motion data mentioned above, it that it may be a audio-visual recording of the user participating in the activity. This information may be used to annotate the mental state information. Annotation may be done manually or automatically, e.g. by the correlator tagging the audio or video data. There may be a time stamp on the EEG recording which correlates with audio/video. In post-performance analysis the EEG output can be synchronised across exactly to what happened at the same time in terms of sporting outcome.

In a further example, the other user-related data 118 may include media information relating to media content (audio and/or video) being consumed at the time of performing the activity.

As discussed above, the central processing unit 106 may be arranged to output data 110 from any one or more of its modules. Where the central processing unit 106 is embodied as a smartphone, the output data 110 may be used to generate a graphical display to be shown on the screen of the smartphone. In other arrangements, the data may be transmitted to another device for storage or display.

The functions of the central processing unit 106 may be all performed on a single device or may be distributed among a plurality of devices. For example, the filter module 112 may be provided on a terminal device (e.g. smartphone) that is communicably connected to the wearable device 102 over a first network, whereas the analyser module 114 may be provided on a separate server computer (e.g. a cloud-based processer) that is communicably connected to the terminal over a second network (which may be a wired network).

FIG. 2 is a schematic view of a portable processing unit 200 that can be used in a wearable sensor that is an embodiment of the invention. The processing unit 200 comprises a substrate 202 on which components are mounted. The substrate 202 may advantageously be made from a flexible material to enable it to fit or conform within the headwear to which the wearable sensor is mounted.

On the substrate 202 there is a processor 204 that control operation of the unit, and a battery 206 for powering the unit. The substrate 202 includes an electrode connection port 208 from which a plurality of connector wires 210 extend to connect each sensor element (not shown) to the processing unit 200. The wearable sensor operates to detect voltage fluctuations at the sensor locations. The processing unit 200 includes an amplification module 212 (e.g. a differential amplifier or the like) for amplifying the voltages seen at the sensors. The amplification module 212 may be shielded to minimise interference.

The processing unit 20 may be configured to take reading from multiple sensors in the array at the same time, e.g. by multiplexing between several channels. In one example, the device may have eight channels, but the invention need not be limited to this number. The voltage fluctuations may be converted to a digital signal by a suitable analog-to-digital converter (ADC) in the processing unit. In one example, a 24-bit ADC is used, although the invention need not be limited to this. The processor 204 may be configured to adjust the number of channels that are used at any given time, e.g. to enable the ADC sampling rate on one or more of the channels to be increased or to switch off channels that have an unusable or invalid output. The ADC sampling rate for eight channels may be 512 Hz, but other frequencies may be used.

The digital signal generated by the processing unit is the EEG signal discussed above. The processing unit 200 includes a transmitter module 214 and antenna 216 for transmitting the EEG signal to the central processing unit. The transmitter module 214 may be any suitable short to medium range transmitter capable of operating over a local network (e.g. a picocell or microcell). In one example, the transmitter module 214 comprises multi band (802.11a/b/g/n) and fast spectrum WiFi with Bluetooth® 4.2 connectivity.

The battery 206 may be a lithium ion battery or similar, which can provide a lifetime equal to or greater than 5 hours for the device. The battery may be rechargeable, e.g. via a port (not shown) mounted on the substrate 202.

The processing unit 200 may be mounted within the fabric of the headwear within which the wearable sensor is mounted. The electrical connection between the sensor elements and the substrate may be via wires as mentioned above, or, advantageously, may be via a flexible conductive fabric. The conductive fabric may be multi-layered, e.g. by having a conductive layer sandwiched between a pair of shield layers. The shield layer may minimise interference. The shield layers may be waterproof or there may further layers to provide waterproofing for the connections. With this arrangement, the wearable sensor can be mounted in a comfortable manner without sacrificing signal security or integrity.

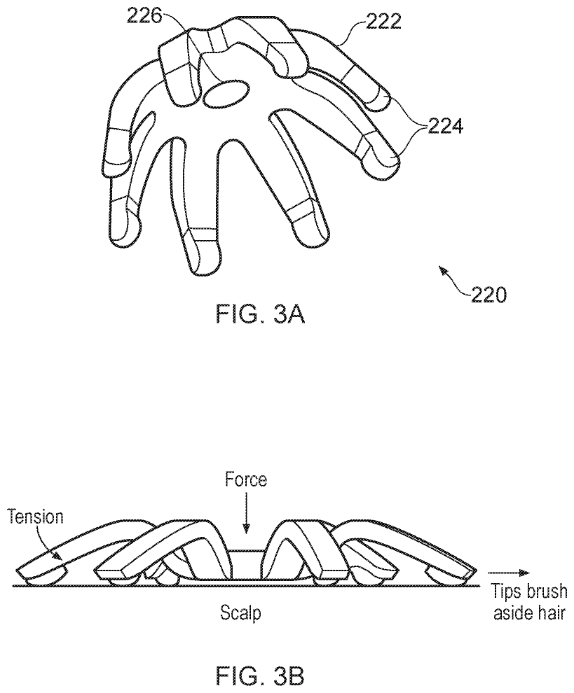

FIG. 3A is a perspective view of an EEG sensor element 220 that can be used in the wearable sensor mentioned above. In this example, the sensor element provides a dry electrode connection to the user's scalp, i.e. the device does not need to be used with a conductive gel or the like. The sensor element 220 comprises a resiliently flexible star-shaped body 222, which may be made from any suitable material, e.g. plastic, graphite, or the like.

The star-shaped body 222 comprises a plurality of legs 224 extending radially outwardly from a central portion. The legs 224 flex outwards as the central portion is pushed onto a surface (e.g. the user's scalp). The end of each leg acts to push aside hair on the scalp to ensure a good physical contact. The legs may have a rubberised tip or the like to improve grip and stability. A conductive micro-electrode 226, e.g. made from gold or similar, is mounted at the central portion of the body to contact the user's scalp when the sensor element is pushed against it. FIG. 3B is a side view of the sensor unit 220 when in contact with a user's scalp. The tension in the legs acts to retain the central portion in contact with the scalp.

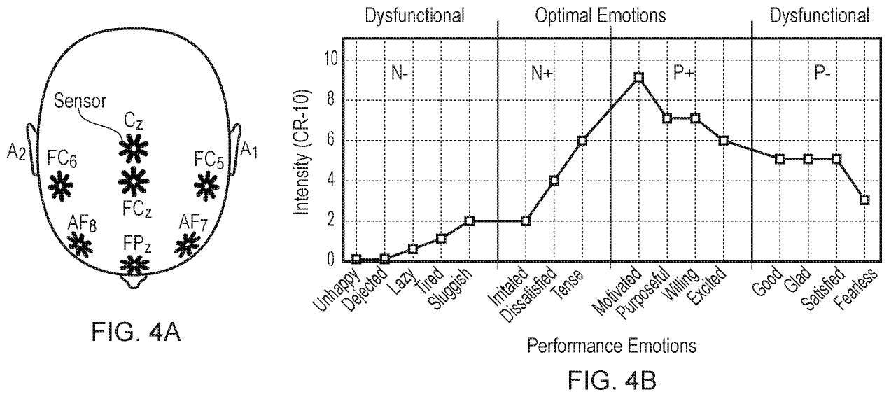

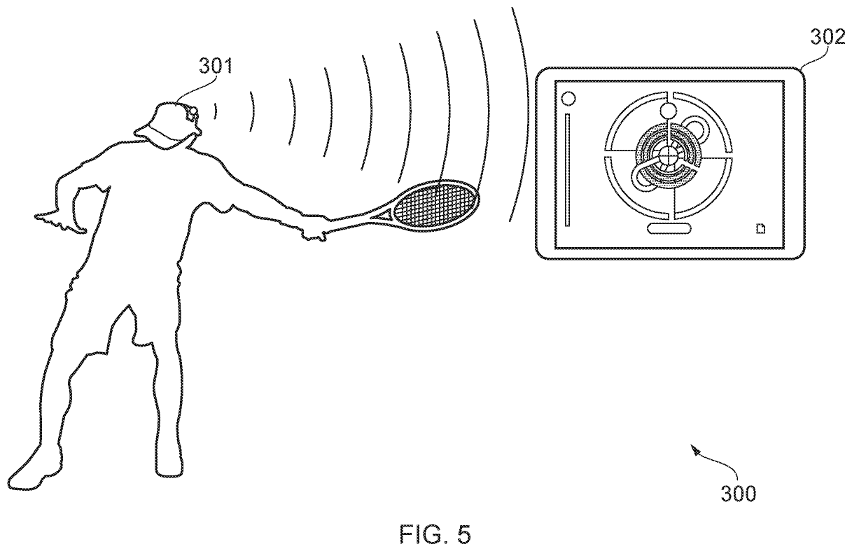

As discussed above, the wearable sensor comprises a plurality of sensor elements arranged in an array over the user's scalp. FIG. 4A is a schematic plan view of a user's head showing the locations of sensor elements such as the one shown in FIG. 3A in such an array. The sensor elements may be placed at nodes recognised under the 10-20 system. In this example, sensor elements are located at the FP_{z}, FC₅, FC₆, C_{z}, AF₇, AF₈ and FC_{z} positions across the frontal lobe.

FIG. 4B shows a chart that may be an example output from the analysis performed by the system discussed above. The chart may assist assessment of an individual zones of optimal functioning by mapping the EEG signal to an intensity value for a set of performance emotions, e.g. by making use of available assessment techniques.

FIG. 5 is a schematic view of an example use environment 300 for the present invention. In this example, the wearable device is a cap 301 worn by a user during an activity (e.g. playing tennis). The cap may be retained on the user's head in a conventional manner, e.g. via an adjustable or elasticated head band. A wearable sensor of the type discussed above may be mounted on or within an inside surface of the cap 301. The sensor array may be located towards the front of the cap to overlie the user's frontal lobe in the manner illustrated in FIG. 4. The processing unit may be located towards the rear or side of the cap.

In this example, the central processing unit is a tablet computer 302 in wireless communication with the wearable sensor over a local network. The tablet computer 302 may have an app installed thereon that provides the functionality discussed above, e.g. filtering and analysing the EEG signal from the wearable sensor, and optionally correlating it with data obtained from other sources. The app provides a graphical user interface that may be arranged to display the output data in a graphical manner.

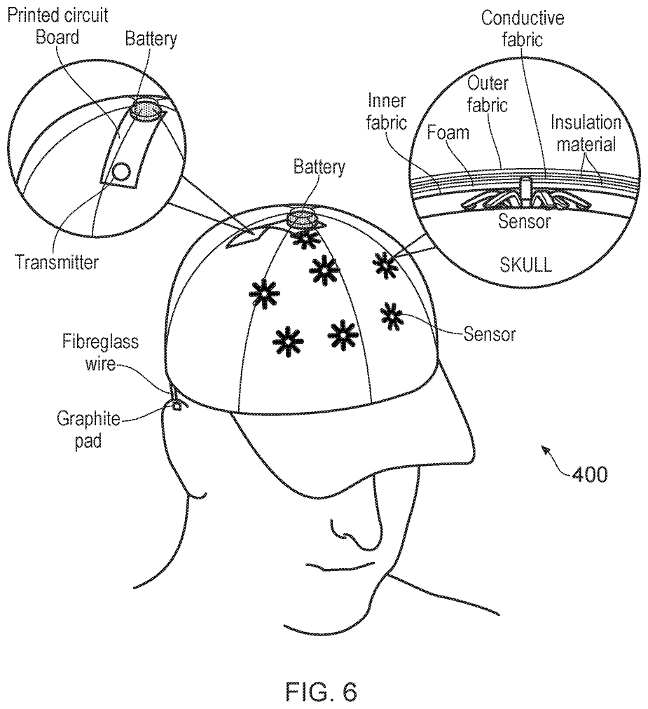

FIG. 6 is a schematic diagram illustrating how the wearable sensor can be mounted in a cap 400. In this example, the processing unit is mounted at the apex of the cap, and curves (or is flexible) to follow the contour of the cap as it extends away from the apex. The interconnections between the sensor elements and the processing unit are fabricated within the cap itself in this example. To achieve this, the material of the cap is a multi-layered structure in which a signal carrying structure is sandwiched between an inner protective layer and an outer protective layer. In this embodiment, the multi-layered structure comprises an inner layer of fabric that is in contact with a user's head. On top of the inner layer of fabric is a layer of foam that protects the user's scalp from unwanted and potentially uncomfortable contact with the conductive layer and processing unit. On top of the layer of foam is an inner insulation layer, a conductive fabric, and an outer insulation layer. The conductive fabric is a flexible electrically conductive material that electrically connects the sensor elements to the processing unit. The inner insulation layer and the outer insulation layer shield the conductive fabric, e.g. to minimise interference with the signals carried by it. Finally an outer fabric layer is provided over the outer insulation layer. The outer fabric may be any conventional durable material used for caps.

In other examples, the sensor elements may be hard wired inside the inner shell.

As shown in the inset of FIG. 6, each sensor element is mounted on the inner fabric layer such that it contacts the user's scalp when the cap is worn. The micro-electrode at the central portion of the sensor element extends though the inner fabric, foam and inner insulation layer to contact the conductive fabric.

A reference electrode is mounted elsewhere on the cap 400 to supply a reference voltage against which the voltage fluctuations are measured. In this example, the reference electrode comprises a graphite pad and fibreglass wire connected to the controller.

A cap such as that shown in FIG. 6 may enable the invention to be used in activities such as golf, tennis, shooting, rowing, archery, sailing, etc.

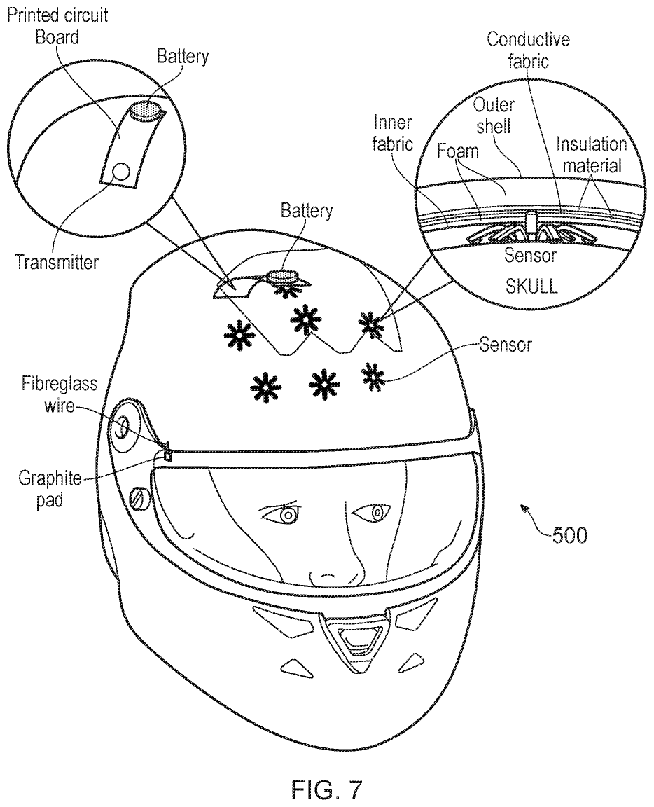

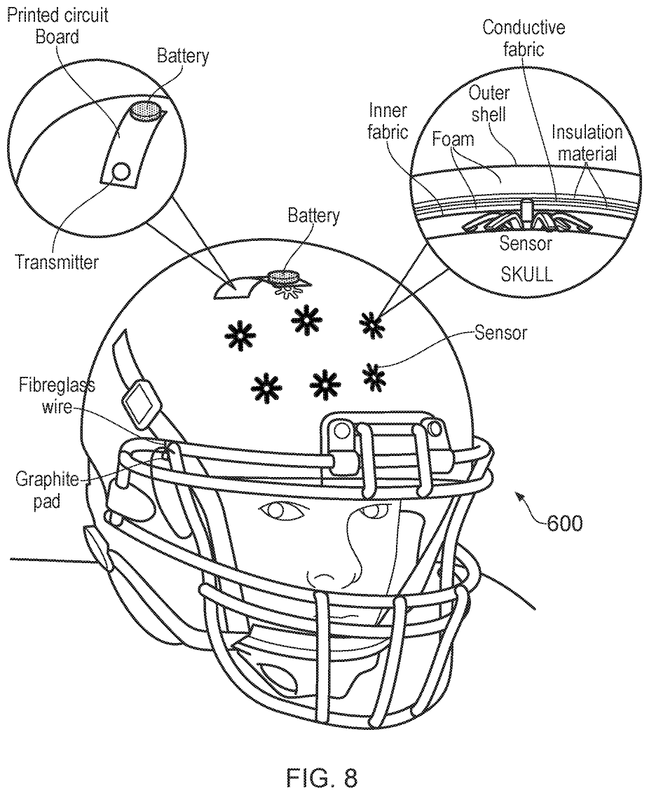

FIG. 7 is a schematic diagram illustrating how the wearable sensor can be mounted in a crash helmet 500. In this example, the processing unit can be mounted either within the main structural shell of the helmet, or outside the shell in in a separate enclosure. The latter arrangement may improve the connectivity of the wearable sensor and may avoid introducing unwanted weaknesses into the structure of the shell.

The sensor array and interconnection to the processing unit may be configured in a similar way to the cap illustrated in FIG. 6. In this example, the multi-layer structure may comprises an inner fabric and inner foam layer similar to those used in FIG. 6. The conductive fabric separated by a pair of insulation layer may be formed on the inner foam layer in a similar manner to that shown in FIG. 6. Above the outer insulation layer there may be an outer foam layer separating the outer insulation layer from the rigid outer shell.

A helmet such as that shown in FIG. 7 may enable the invention to be used in activities such as motor sport, alpine sport, cycling, etc.

Other types of protective headgear are worn by users participating in other sports events or training. For example, specific types of headgear may be worn when playing rugby, hockey (especially ice hockey), American football, cricket, baseball and the like. FIG. 7 is a schematic diagram illustrating how the wearable sensor can be mounted in a sports helmet 600. The integration of the wearable sensor into the sports helmet 600 is done in a similar way to the crash helmet 500 and is not described again.

In another example, the processing unit may be encased or encapsulating in waterproof material any mounted within a swimming cap or the like.

The system discussed above provides a readily accessible means for a user to understand and utilise the mental states experiences during an activity. The output data from the system may represent biofeedback (i.e. neurofeedback) that in turn can be used to train the user in a manner to improve their performance. It is recognised in the field of sport, and especially elite sport, that there is benefit in honing emotional intelligence and cognitive resilience before they are tested in competition. The wearable sensor of the invention may be particular suitable for measuring a signal indicative of fear/anxiety and confidence/excitement, which are understood to have a polarising effect on athletic performance.

By integrating the sensor into conventional sportswear, the system is able to provide biofeedback in a repeatable manner for user's actually engaged in real-world performance. The results may be used to as part of a neurofeedback programme to improve or optimise the user's performance. For example, where the EEG signals are recorded in configuration with audio-visual data of the user performing the activity, the user may have the output played back to them to train emotional “muscle memory”, e.g. to encourage repetition of optimal performance.

As mentioned above, the system of the invention may be configured to operate in conjunction with other wearable devices that measure biometric data. In one example, the system may be configured to interface with the HealthKit and ResearchKit frameworks released by Apple Inc.

Although the examples above present a single wearable sensor configuration, it can be understood that the invention may be implemented in a variety of ways that still provide the advantages set out herein. For example, in practice there may be a range of wearable products with different levels of functionality to suit different markets. A wearable device for an amateur athlete interested in self-improvement may for example have a sensor array with fewer sensor elements than a wearable device targeted at an elite athlete who has their own dedicated training staff.

The discussion above mentions use of the device in the context of performing a sporting activity. However, it can be understood that the term “activity” used herein has a broader reach, and may encompass fitness assessment activities, e.g. for military selection, health insurance or rehabilitation purposes. The invention may also find application in other fields, e.g. to interpret emotional reaction to media in return for discounted streaming services.

1. A biofeedback system comprising:
a wearable sensor comprising:
a sensor array detecting an electroencephalographic (EEG) signal from a user wearing the wearable sensor; and
a communication unit wirelessly transmitting the EEG signal; and
a central processing unit arranged to receive the EEG signal transmitted from the wearable sensor, the central processing unit comprising an analyser module arranged to generate, based on the EEG signal, output data that is indicative of mental state information of the user,
wherein the wearable sensor is incorporated into headgear worn by the user during participation in an activity, whereby the output data provides real-time mental state information for the user whilst performing the activity.

2. The biofeedback system according to claim 1, wherein the headgear is specific to the activity to be performed

3. The biofeedback system according to claim 1, wherein the headgear comprises any of a baseball cap, a crash helmet, a sport helmet, and a swimming cap.

4. The biofeedback system according to claim 1 comprising a display arranged to present a graphical representation of the output data.

5. The biofeedback system according to claim 1, wherein the central processing unit is part of a portable computing device.

6. The biofeedback system according to claim 1, wherein the central processing unit comprises a filter module removing unwanted frequencies from the EEG signal before it is used to generate the output data.

7. The biofeedback system according to claim 1, wherein the central processing unit is arranged to receive biometric data for the user concurrently with the EEG signal, and
wherein the analyser module is arranged to generate the output data based on the EEG signal and the biometric data.

8. The biofeedback system according to claim 7, wherein the biometric data includes any one or more of breathing patterns, heart rate, blood pressure, skin temperature, galvanic skin response, and salivary cortisol.

9. The biofeedback system according to claim 7, wherein the central processing unit comprises a correlator module arranged to correlate the received biometric data with the EEG signal.

10. The biofeedback system according to claim 1, wherein the output data relates the EEG signal to an individual zone of optimal functioning (IZOF) model for the user.

11. The biofeedback system according to claim 1 wherein the wearable sensor comprises:
headgear to be worn by a user while participating in an activity, wherein the sensor array is mounted in the headgear;
a detector mounted in the headgear, the detector being arranged to detect voltage fluctuations at each of the plurality of sensor elements and generate an EEG signal therefrom; and
a transmitter wirelessly transmitting the EEG signal to a remote device for analysis,
wherein the plurality of sensor elements are disposed within the headgear to contact the scalp of the user when the headgear is worn.

12. The biofeedback system according to claim 11, wherein the detector and transmitter are mounted on a flexible substrate that conforms to the shape of the headgear.

13. The biofeedback system according to claim 11 including a conductive interconnection structure formed within the headgear to provide an electrical connection between the sensor array and the detector.

14. The biofeedback system according to claim 13, wherein the conductive interconnection structure comprises a conductive fabric sandwiched between a pair of insulation layers.

15. The biofeedback system according to claim 13, wherein the conductive interconnection structure is encased within the material of the headgear.

16. The biofeedback system according to claim 11, wherein the plurality of sensor elements are disposed within the headgear to lie across a frontal lobe of the user when the headgear is worn.

17. The biofeedback system according to claim 11, wherein the plurality of sensor elements are located at FP_{z}, FC₅, FC₆, C_{z}, AF₇, AF₈ and FC_{z} positions across the frontal lobe.

18. The biofeedback system according to claim 11, wherein each sensor element comprises a star-shaped body having a plurality of resiliently deformable legs that extend radially from a central portion.

19. The biofeedback system according to claim 18, wherein the central portion of the star-shaped body is electrically conductive.

20. The biofeedback system according to claim 1, wherein the headgear is specific to the activity to be performed.
